(19) United States
(12) Patent Application Publication
(10) Pub. No.: US 2002/0025324 A1
(43) Pub. Date: Feb. 28, 2002

(54) RECOMBINANT BIVALENT BACTERIAL VACCINE FOR THE PREVENTION OF MASTITIS

(76) Inventor: JONATHAN R. SEALS, UPTON, MA (US)

Correspondence Address: HOLLIE L BAKER, HALE AND DORR, 60 STATE STREET, BOSTON, MA, 02109

(21) Appl. No.: 08/901,062

(22) Filed: Jul. 25, 1997

Related U.S. Application Data
(60) Provisional application No. 60/043,824, filed on Apr. 14, 1997.

(51) Int. Cl. A61K 39/00; A61K 39/38; A61K 39/02

(52) U.S. Cl. 424/200.1; 424/184.1

(57) ABSTRACT

This invention concerns a recombinant vaccine for the prevention of mastitis in milking mammals, particularly cows, and to methods for the production and use of the vaccine. The vaccine against mastitis is made from an E. coli cell that has been genetically engineered, or transformed, to contain and express an antigen from one or more of the pathogens responsible for causing mastitis.

### FIELD OF THE INVENTION

This invention is directed to a recombinant bivalent bacterial vaccine for the prevention of mastitis in milking animals, especially in dairy cows.

### BACKGROUND OF THE INVENTION

Mastitis is an infection or inflammation of the mammary gland, particularly important in dairy cattle. Although there are many pathogens associated with mastitis, the primary pathogens which cause the infection are Staphylococcus aureus, which are involved in approximately 30-50% of infections, Escherichia coli, which are involved in approximately 30-40% of infections, and Streptococcus sp., which are involved in approximately 20-40% of infections.

Infection of the mammary gland in dairy cows is a major health problem for the dairy industry. Chronic mastitis in a milking cow produces an abnormal udder which, when the udder is palpated, feels hard to the touch. Once infected, milk quality declines, with milk produced from infected cows having an increased somatic cell count, and overall milk production is reduced. In addition, with infections caused by E. coli, or other gram negative bacteria, the cow may suffer from endotoxemia, a potentially fatal condition.

Conventional treatment for mastitis includes sanitary milking conditions and administration of antibiotics to the infected cow. While antibiotics are being administered, however, the milk produced from this cow will contain residual antibiotics, and therefore must be discarded.

Thus, in the dairy industry, the annual cost to the farmer of cattle with mastitis includes a lower price for lower quality milk, reduced milk production, discarded milk, treatment for the infected cow, and cow replacement costs. Accordingly, there continues to be an industry-wide need for a vaccine for the treatment and prevention of mastitis.

### SUMMARY OF THE INVENTION

This invention concerns a recombinant vaccine for the prevention of mastitis in milking mammals, particularly cows, horses, and goats. It has been found that an effective vaccine against mastitis can be made from an E. coli cell that has been genetically engineered, or transformed, to contain and express an antigen from one or more of the pathogens responsible for causing mastitis. In the preferred embodiment, the vaccine also incorporates an adjuvant. This invention is also directed to the production of the vaccine and to its uses.

### BRIEF DESCRIPTION OF THE FIGURES

FIG. 1 is a diagram of a temperature-inducible, ampicillin-resistant expression vector, pXAFB1, containing the DNA coding region for fibronectin binding protein B from S. aureus.

FIG. 2 is a graphic depiction of the E. coli antibody titer produced by the vaccine of this invention and a commercially available vaccine.

FIG. 3 is a graphic depiction of a comparison of clinical disease symptoms between vaccinated cows and unvaccinated control cows after mastitis challenge.

FIG. 4 is graph of the somatic cell count in milk from vaccinated cows and unvaccinated cows after mastitis challenge.

### DETAILED DESCRIPTION OF THE INVENTION

The vaccine of this invention includes an E. coli cell that is modified by genetic engineering, or recombinant techniques, so that it is capable of expressing a gene, or a gene sequence, of an antigen from one or more of the pathogens responsible for causing mastitis. As E. coli is one of the pathogens that is associated with mastitis, the antigen chosen for expression in the E. coli cell is from a second pathogen, such as S. aureus or Streptococcus sp. By transforming an E. coli cell with the gene, or gene sequences, of an antigen from a second pathogen, the vaccine of this invention will be bivalent, that is, it will elicit an immune response against two of the pathogens associated with mastitis. In addition, the E. coli cell can be transformed with a genetically engineered construct that contains gene sequences for more than one antigen that will be expressed by the E. coli host cell. In this type of vaccine, the vaccine of this invention will be trivalent by eliciting an immune response against three of the pathogens associated with mastitis.

In another embodiment, E. coli cells are transformed with an antigen of one pathogen responsible for causing mastitis, and, separate E. coli cells are transformed with a different antigen of the same pathogen or with an antigen of a different pathogen responsible for causing mastitis. According to this invention, after expression of the genes coding for the antigens, and recovery of the E. coli cells and expressed antigen, the two transformed E. coli and expressed antigen components are mixed together to form a single vaccine for administration.

Any suitable E. coli can be used as a host cells for the vaccine of this invention. E. coli host cells are well known in the art, as are appropriate vectors that can be used for cloning and expressing the antigen of choice. General techniques for selection of suitable E. coli host cells, vectors, nucleic acid manipulation, transformed host cell selection, incubation, induction, and the like, useful for the practice of the claimed invention are well known and are described generally, for example, in Sambrook et al., “Molecular Cloning: A Laboratory Manual”, Vols. 1-3 (Cold Spring Harbor Laboratory Press, 2 ed., 1989) or in Ausubel et al., Current Protocols in Molecular Biology (Green Publishing and Wiley-Interscience: New York, 1987).

The bacterial host cell of choice for expression of recombinant DNA has long been Escherichia coli. The disadvantages of this microorganism in genetic engineering is an advantage to its use in the vaccine of this invention. Transformed E. coli lacks the inability to secrete recombinantly produced proteins, so that recombinantly produced proteins are precipitated into inclusion bodies within the cell. In addition, it is difficult to purify a recombinantly produced protein from E. coli.

The vaccine of this invention is advantageous in that, although it is a genetically engineered or recombinant product, no protein purification is necessary, or required. In this vaccine, the recombinant E. coli host cell is also a component of the vaccine, hence, if a gene from a different pathogen is expressed, it is a bivalent bacterial vaccine. In addition, the vaccine of this invention is advantageous in the relatively low cost of production, which is an important consideration in the veterinary medicine field.

The gene or gene sequences of an antigen from a pathogen that can elicit an immune response can be chosen from the known pathogens. For example, many genes and gene sequences of the Streptococcus family of pathogens have been described, such as those described in, but not limited to, U.S. Pat. Nos. 4,695,562; 4,705,684; 5,302,386; 5,610,011; and 5,648,241, incorporated herein by reference.

Fibronectin binding protein is a surface protein from S. aureus which may serve as the expressed antigen component of the bivalent vaccine of this invention against S. aureus infections. Fibronectin binding protein is well known in the art and, as the term is used herein, can include any portion of the gene coding for protein that is capable of eliciting an immune response, including the gene A (fibronectin protein A) and gene B (fibronectin protein B). The identification, isolation, and sequencing of fibronectin binding protein, and its components, from S. aureus, are described in detail, for example, U.S. Pat. Nos. 5,175,096; 5,189,015; 5,320,951; 5,440,014; and 5,571,514, which are incorporated herein by reference.

The vaccine is preferably administered with an adjuvant, with the preferred adjuvant being a saponin, as described in U.S. Pat. Nos. 5,057,540; 5,273,965; and 5,443,829, and PCT/US96/19252, incorporated herein by reference. The most preferred is a saponin described as QS-21, which is described in U.S. Pat. No. 5,057,540.

The vaccine is produced from a recombinant E. coli culture which has been induced to express the antigen of a pathogen associated with mastitis. The E. coli cells are recovered from the culture using any suitable technique, typically by centrifugation which recovers the cells as a paste. The cell paste will also contain the expressed antigen. The cells are then inactivated by incubation at a temperature and for a time sufficient to produce inactivated, or heat-killed, cells. The inactivated cells, which are termed bacterin in the art, are recovered and suspended in an appropriate pharmacological carrier. The carrier may also contain one or more preservatives. Further, the vaccine will preferably be mixed with an adjuvant.

The recombinant bivalent bacterial vaccine will contain a biologically effective concentration of the inactivated E. coli and expressed antigen. A biologically effective concentration will typically be from about 0.5 to about 2.0 mg/ml of inactivated E. coli and the expressed antigen. One skilled in the art will be able to determine if this component of the vaccine of this invention should be increased or decreased, depending upon the levels of expression of the selected antigen and the immunogenicity of the selected E. coli host cell. In addition, in the preferred embodiment, the vaccine of this invention will contain an adjuvant, preferably a saponin adjuvant, more preferred QS-21. The amount of adjuvant can be determined through routine experimentation, but will typically be from about 0.1 to about 1.0 mg/ml of adjuvant, preferably from about 0.1 to about 0.5 mg/ml. The vaccine of this invention will be in a pharmacologically acceptable solution, typically a saline solution such as a phosphate-buffered saline solution, and may be preserved with a known preservative, such as phenol or glutaraldehyde.

The timing of the vaccine of this invention is preferably close to calving.

Typically the vaccine will be administered within thirty days or less prior to calving, and in addition, is preferably administered after calving. The vaccine can be administered more than once prior to calving and more than once after calving, depending upon the degree of immunogenicity which is exhibited after each administration. One skilled in the art can determine the timing of the administration of the vaccine and the number of times the vaccine can be administered for each vaccine of this invention through routine screening involving mastitis challenge.

The route of administration of the vaccine is not critical, and may be by subcutaneous, intracutaneous, and intramuscular injection. Finally, it will be appreciated by those skilled in the this field that the bivalent vaccine of this invention may be combined with vaccines of other genera of bacteria to provide a single broad spectrum vaccine.

The following examples describe in detail the preparation and administration of one embodiment of this invention comprising a vaccine made from an E. coli cell genetically engineered to express a fibronectin binding protein and containing the adjuvant QS-21. These examples are provided to illustrate the vaccine of this invention and the invention is not intended to be limited to these specific illustrations.

### EXAMPLE 1

### Vaccine Immunogen

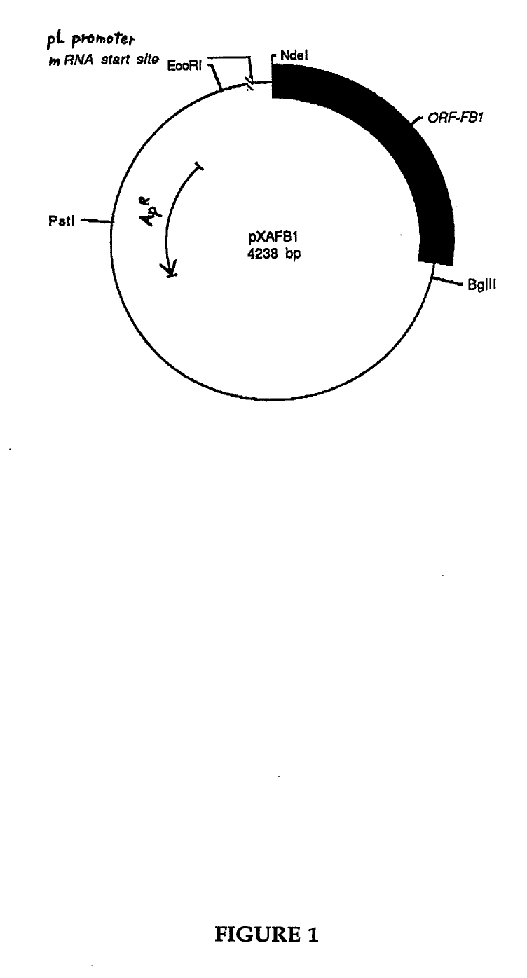

A temperature-inducible, ampicillin-resistant expression vector, pXAFB1, which is diagramed in FIG. 1, containing the DNA coding region for a protein of 388 amino acids, comprising a portion of fibronectin binding protein B from S. aureus, was transfected into E. coli strain MZ-1. The amino acid sequence (FB1) is shown in SEQ ID NO. 1 and the nucleotide sequence is shown in SEQ ID NO. 2. The recombinant host cells were isolated by antibiotic selection.

### EXAMPLE 2

### Vaccine Preparation

Recombinant E. coli was cultured in growth medium at 37° C., then incubated at 42° C. for 2 hours to induce recombinant protein expression. The details of the vaccine preparation are given in Table 1.

TABLE 1

| | |
|---|---|
| (a) | 250 microliters MX1/pXAFB1 was placed with 400 mls culture |
| | media and incubated at 32° C. for 2 hours then at room temperature |
| | for 8 hours. |
| (b) | A 16 L vessel was inoculated with 800 mls culture, air flow at |
| | 16 L/min and rpm varied to maintain DO of 40%. |
| (c) | Post 9-10 hours rpm set to 400 and manually mixed in pure O₂ to |
| | maintain DO of 40% with L/min total gas of 16. |
| (d) | 1L media bolus injected within OD range of 20-24. |
| (e) | Induction temperature of 38° C. initiated within OD range of 26-30. |
| (f) | Induction maintained for 90 minutes, then cooled to 15-20° C., while |
| | cooling, set to low agitation and low-air flow. |
| (g) | Harvested in continuous flow centrifuge at approximately 12 k g's, 4- |
| | 15° C., and 20-22 L/hr. |
| (h) | Cell paste washed in 1:25 PBS and recovered in centrifuge. |

After the E. coli cell paste was recovered by centrifugation, it was then washed by resuspension and centrifugation. Cells were resuspended in 0.1% glutaraldehyde and incubated at 37° C. for 1 hour. The killed cells (bacterin) were recovered by centrifugation, resuspended in PBS, pH 6.5, and assayed for protein content by BCA assay. The bacterin was diluted in PBS and mixed with QS-21 adjuvant and preservative to concentrations shown below in Table 2.

TABLE 2

| | |
|---|---|
| 1. | 16 L Batch Fermentation produced approximately 1 kg of cell paste |
| | (1 g cell paste yields 75 mg bacterial protein). |
| 2. | Weigh out 24 g of cell paste in sterile petri dish on lab balance and |
| | transfer to sterile centrifuge bottle using sterile cell scraper. |
| 3. | Resuspend cell paste with 300 mL of PBS pH 7.2 in sterile centrifuge |
| | bottle (400 mL cap.) using over head stirrer at setting 32 for 15 |
| | minutes. Split into 2x 150 mL aliquots then bring to 300 mL using |
| | PBS pH 7.2. (1g of cellpellet: 25 mL of buffer). |
| 4. | Centrifuge at 6,000 rpm (g = 6084) for 20 minutes at 20° C. Decant |
| | supernate into waste beaker. |
| 5. | Resuspend pellets in PBS pH 7.2 (300 mL/centrifuge bottle) |
| 6. | Split into 2x 300 mL aliquots and place into sterile polycarbonate |
| | flasks and cover flask openings. |
| 7. | Add 1200 μL of 25% solution of glutaraldehyde to each flask |
| | (glutaraldehyde at 0.1%, v/v). Incubate in shaker water bath at 37° C. |
| | for 1 hour. |
| 8. | Combine contents of both flasks into one flask by pipeting. Split into |
| | 2x 300 mL aliquots in 400 mL sterile centrifuge bottles. Centrifuge |
| | at 6,000 rpm (g = 6084) for 20 minutes at 20° C. Decant supernate |
| | into waste beaker. |
| 9. | Resuspend pellet in 450 mL of PBS pH 6.5. (Estimated 4 mg of |
| | protein/mL of buffer in 450 mL). (BCA estimate 5 mg/mL). |
| 10. | Store at 2-8° C. |

### EXAMPLE 3

### Vaccine Composition

The vaccine composition was made from the following components:

(a) 0.5-2.0 mg/ml of inactivated E. coli genetically engineered to express recombinant construct of S. aureus fibronectin binding protein;

(b) 0.1-0.5 mg/ml of QS-21 adjuvant;

(c) Phosphate-buffered saline, pH 6.5; and,

(d) 0.05% phenol or glutaraldehyde as preservative.

### EXAMPLE 4

### Immunization Schedule

Holstein dairy cows were administered two or three doses, each consisting of 2 ml of vaccine, injected subcutaneously to the right of the tail-bone. The three-dose schedule consisted of immunizations 30 and 18 days pre-calving, and 18 days post-calving. The two-dose schedule consisted of immunizations 18 days pre-and 18 days post-calving.

In addition, for comparison purposes, a commercially available vaccine, which has the name “J.VAC” a Trademark of Sanofi, was administered to cows according to the immunization schedule provided by Sanofi. “J.VAC” is an E. coli bacterin-toxoid vaccine.

### EXAMPLE 5

### Vaccine Immunogenicity

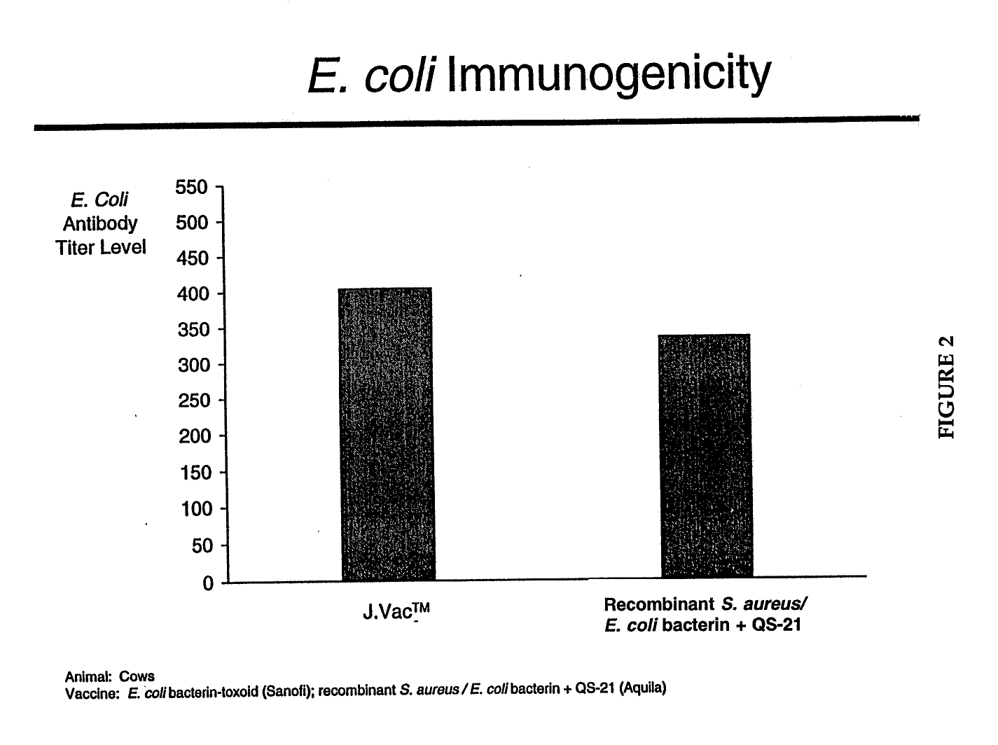

Sera were collected from cows before and after each immunization; antibody titers were determined by ELISA assays using killed MZ-1 E. coli or purified FB1 protein as antigens. The results are depicted in FIG. 2, which shows E. coli antibody titer levels between “J.VAC” and the vaccine of this invention.

### EXAMPLE 6

### Vaccine Efficacy

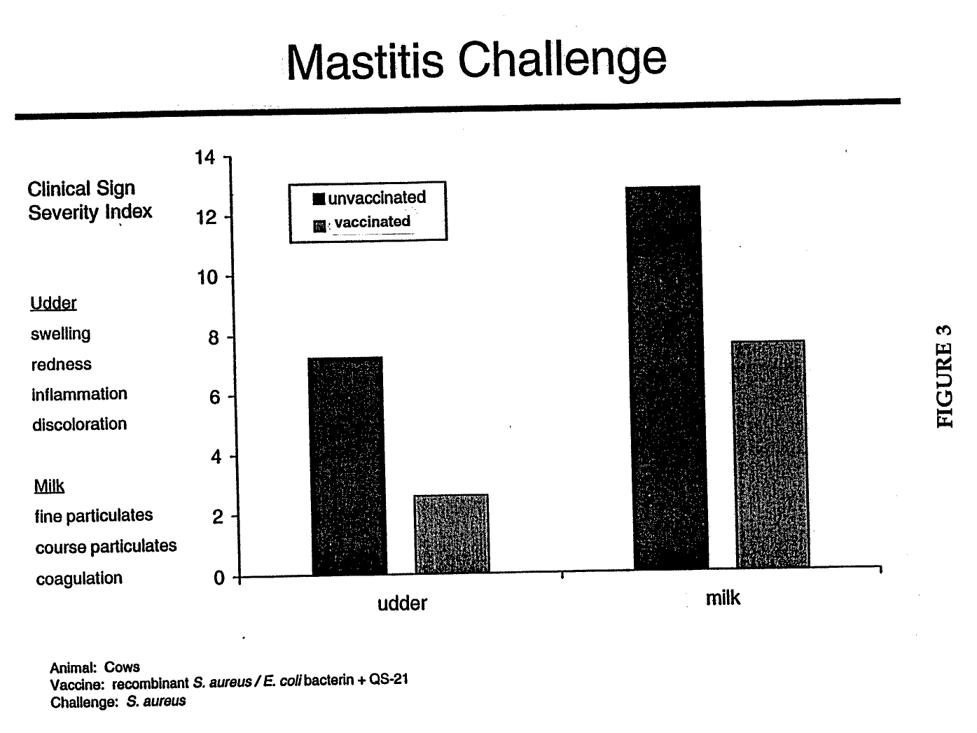

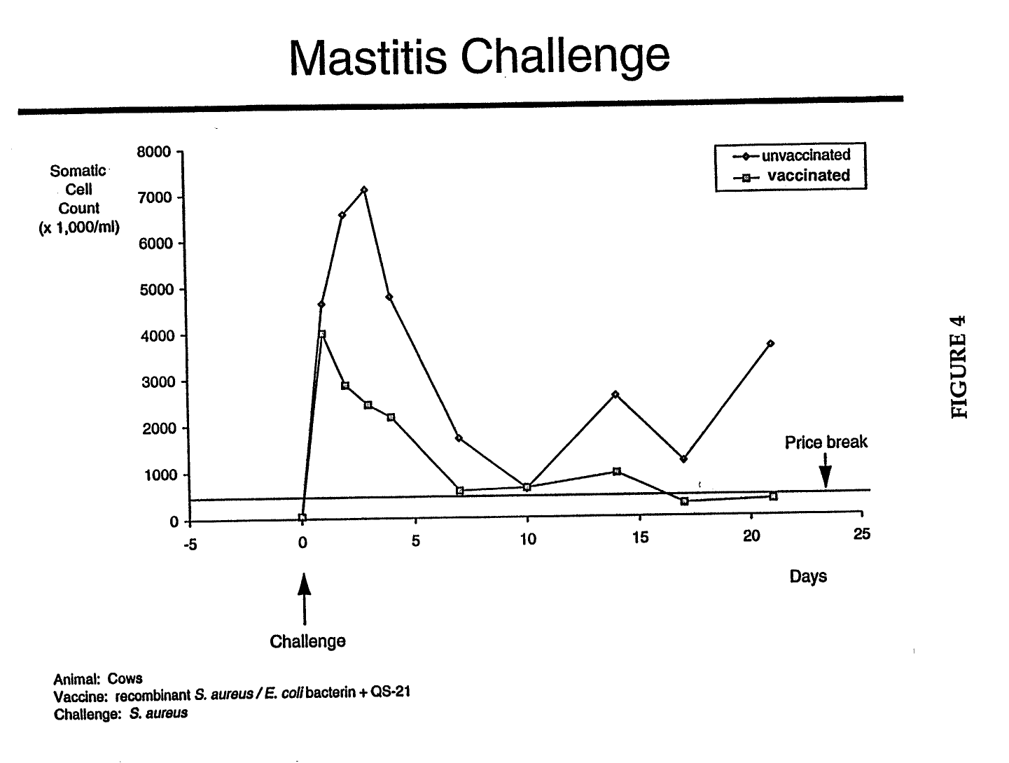

S. aureus mastitis was experimentally induced in cows 5-10 weeks after calving by administration of S. aureus through the teat orifice. This method is described in Table 3. The course of disease was followed and compared in vaccinated and unvaccinated animals. The results of this comparative testing is shown in FIGS. 3 and 4.

TABLE 3
S. aureus Mastitis Challenge Protocol

| | |
|---|---|
| (a) | Second to fourth lactation cows |
| (b) | 2-4 weeks after calving |
| (c) | Serum and milk samples taken pre-challenge |
| (d) | Challenge organism: S. aureus strain Newbold 305 |
| (e) | Challenge stock grown in BHL broth, diluted in sterile milk |
| (f) | Challenge administered after morning milking |
| (g) | Each quarter a separate challenge site |
| (h) | 200 ul of challenge stock deposited into teat via 15 mm needle |
| (i) | Challenge dose of >40 cfu sufficient to cause infection in all quarters |
| | (Dose estimated by absorbance on day of challenge; determined by |
| | plating after challenge; target range = 50-100 cfu; actual range in |
| | trials = 42-187 cfu) |
| (j) | Cow not milked until evening |
| (k) | Cows monitored for: |

Clinical signs (general observations, temperature, udder palpation, milk appearance)

California Mastitis Test (CMT)

Milk Somatic Cell Count (SCC)

S. aureus in milk by culture on blood agar plates

The invention has been described in some detail by the foregoing examples, which are intended to merely illustrate the invention. The scope of the invention is not to be considered limited thereto.

What is claimed is:

1. A vaccine comprising:
(a) inactivated, transformed E. coli cells which have expressed a gene coding for an antigen from a pathogen associated with mastitis; and,
(b) a pharmacological carrier.

2. The vaccine of claim 1, further comprising an adjuvant.

3. The vaccine of claim 2, wherein the adjuvant is saponin.

4. The vaccine of claim 3, wherein the saponin adjuvant is QS-21.

5. The vaccine of claim 1, wherein the antigen is from Staphylococcus aureus.

6. The vaccine of claim 5, wherein the antigen is a fibronectin binding protein.

7. The vaccine of claim 1, wherein the antigen is from Streptococcus.

8. A vaccine comprising:
(a) inactivated, transformed E. coli cells which have expressed a gene coding for fibronectin binding protein in a biologically effective concentration of from about 05 to about 2.0 mg/ml;
(b) a saponin adjuvant; and,
(c) a pharmacological carrier.

9. The vaccine of claim 8, wherein the saponin adjuvant is QS-21.

10. A vaccine produced according to the process comprising the steps of:
(a) culturing E. coli cells transformed with a gene coding for an antigen from a pathogen associated with mastitis;
(b) expressing the gene coding for the antigen to produce the antigen;
(c) recovering the E. coli cells and expressed antigen;
(d) inactivating the recovered E. coli cells;
(e) incorporating the inactivated E. coli cells and expressed antigen in a biologically effective concentration in an appropriate pharmacological carrier.

11. The vaccine of claim 10, further comprising the step of admixing an adjuvant in the pharmacological carrier.

12. The vaccine of claim 11, wherein the adjuvant is saponin.

13. The vaccine of claim 12, wherein the saponin adjuvant is QS-21.

14. The vaccine of claim 10, further comprising the step of admixing a preservative in the pharmacological carrier.

15. The vaccine of claim 14, wherein the preservative is phenol or glutaraldehyde.

16. The vaccine of claim 10, wherein the pharmacological carrier is a saline solution.

17. The method of vaccinating a cow against mastitis, comprising administering to the cow an effective amount of the vaccine of any of claims 1-9.
